(19) United States
(12) Patent Application Publication
(10) Pub. No.: US 2004/0226392 A1
(43) Pub. Date: Nov. 18, 2004

(54) Apparatus for detecting and reporting environmental conditions in bulk processing and handling of goods

(75) Inventor: Wayd A. McNally, Stratford (CA)

Correspondence Address: MARKS & CLERK, P.O. BOX 957, STATION B, OTTAWA, ON, K1P 5S7, CA

(73) Assignee: Sensor Wireless Incorporated

(21) Appl. No.: 10/796,042

(22) Filed: Mar. 10, 2004

Related U.S. Application Data
(60) Provisional application No. 60/452,967, filed on Mar. 10, 2003.

(51) Int. Cl. G01N 33/00; G01M 19/00; G01N 33/02

(52) U.S. Cl. 73/866.1; 702/189

(57) ABSTRACT

An apparatus for wireless instant real-time measurement of processing and handling produce and goods is presented. Electronic sensors monitor various physical parameters, sensor output measurements are analyzed, and reports are conveyed wirelessly in real-time to a remote display device for instant display and further analysis. Measurement profiles provided prove useful in determining optimum operating parameters ensuring high efficiencies in processing, storing, transport, and handling of produce and goods. Multiple sensor output measurements are combined to derive operating parameters which are reported along side sensor output measurements. Processing sensor output measurements also enables raising alarms when environmental extremes are experienced. The monitoring, detection, measurement and gathering of the environmental conditions data is found to be important in preventative maintenance, handling efficiency and performance monitoring in food safety and quality programs. The apparatus provides handlers with directed information on what processing and handling issues to address to recover revenues previously accepted as lost product and packaging.

### FIELD OF THE INVENTION

The invention relates to wireless detection of environmental parameters effectual in quality and efficiency of bulk processing and handling of goods, and in particular to apparatus and methods for detecting, measuring, and wireless reporting of environmental conditions to which goods are exposed to in bulk processing and handling thereof.

### BACKGROUND OF THE INVENTION

Methods and apparatus which measure environmental conditions experienced by produce during harvesting; produce and general goods during sorting, cleaning, packaging, and other handling and processing operations, are necessary to determine the extent to which the produce and the goods may be expected to incur damage, if damaged at all.

In the field it is known to inspect processed and handled goods.

A prior art U.S. Pat. No. 3,656,352 entitled “Impact Monitoring Apparatus” which issued on April 18^{th}, 1972 to Low et al. describes a method to implementing a rudimentary accelerometer for use in controlled testing environments. The proposed approach is a variation on a much older technique to measure acceleration by allowing a suspended mass to cause an arm, or similar device, to bend/deflect. The length of the arm and the mass, as well as the applied acceleration determines the amount of deflection of the arm. The amount of deflection of the arm can be measured by a variety of techniques, typically capacitive or resistive sensing elements. The controlled testing technique proposed suffers due to a lot of signal conditioning required to provide a usable output signal.

Currently many off-the-shelf accelerometers are based on a similar principle and include all the post-processing electronics required to output a voltage corresponding to a level of impact. It is not be feasible to employ this technology in a small enough package to inspect bulk processing and handling of goods.

The size of the measuring device is important. Particularly, impact measuring solutions designed based on the assumption that the measurement device is “point-sized”: small enough that impacts occurring at different points on the surface thereof will register the same, have been found to be inadequate. The assumption is inaccurate for larger products/goods, and also inaccurate form relatively small products/goods yet having a relatively high ratio of length-to-cross-section diameter, such as a small vial.

Another prior art U.S. Pat. No. 4,745,564 entitled “Impact Detection Apparatus” which issued on May 17^{th}, 1988 to Tennes et al. describes a device which is only capable to record data when the measured impact level reaches a certain threshold. One of the main reasons for detecting, measuring and reporting environmental conditions to which goods are exposed, to in bulk processing and handling, is to prevent loss of goods. Typically there is a gradual ramp-up to (equipment) problems/failures and therefore there is a need for continuous monitoring, detection, measurement, and reporting of environmental conditions. The storage of data for later retrieval presented in the Tennes et al.′ device, makes it difficult to determine, from the time stamp, the location, along a processing line, where the above threshold event occurred. It is not always the case that goods move at constant speed in a processing line, which the Tennes et al. proposed solution assumes.

Another prior art U.S. Pat. No. 4,829,812 entitled “Device for Assessing Processing Stress” which issued on May 16, 1989 to Parks et al. describes a device for assessing stress in mechanical processing of agricultural or manufactured products. The embodiments of the device presented have only eight unidirectional levels of impact detection which have been found insufficient for bulk processing and handling. In the device described by Parks et al. the sensor itself takes a lot of space in the device thus making it impossible to use several sensors and/or to place them at selected locations. The Parks et al. device only records the highest impact in it's eight-position threshold windows for a given period of time which can only provide a “complies/does not comply” assessment without correlation between experienced events and the inspected processing apparatus.

Another prior art U.S. Pat. No. 5,426,595 entitled “Portable Autonomous Device for the Detection and Recording of Randomly Occurring Phenomena of Short Duration” which issued on June 20^{th}, 1995, to Picard describes a device which measures, only infrequent, data that surpasses a threshold level. Picard's device is designed for measuring impact during long-term handling such as shipping. Similarly to the Parks et al. device, the Picard device can only provide a “complies/does not comply” assessment without correlation between experienced events and the inspected handling experience.

Another prior art U.S. Pat. No. 5,811,680 entitled “Method and Apparatus for Testing the Quality of Fruit” which issued on Sep. 22^{nd}, 1998 to Galili et al. describes a device that imparts a controlled force on the surface of a fruit and measures the strain on the fruit, or deflection of the fruit's skin. While it is important to determine the ability of fruit to withstand forces, this type of fruit testing does not answer questions related to the monitoring and detection of environmental conditions experienced in processing and handling of goods, nor can this type of testing be used to determine the location, in a processing line, where produce/goods experience forces which cannot be withstood.

Yet another prior art U.S. Pat. No. 6,125,686 entitled “Impact Measuring Device for Delicate and Fragile Articles” which issued on Oct. 3^{rd}, 2000 to Thomas Haan, and to the present inventor Wayd McNally, describes a rudimentary apparatus for continuous monitoring of unidirectional impact experienced by articles being processed and/or handled. However it has been found, in practice, that unidirectional impact measurement alone cannot account for all failures experienced by equipment used in handling/processing articles. Some loss of products/goods is incurred when more than one environmental condition, having a cooperative effect, are encountered.

There therefore is a need to solve the above mentioned issues.

### SUMMARY OF THE INVENTION

In accordance with an aspect of the invention, a apparatus for wireless detection of environmental parameters effectual in quality and efficiency of bulk processing and handling of goods is provided.

In accordance with a further aspect of the invention, the apparatus comprises means for concurrently detecting, measuring and transmitting a group of disparate environmental conditions to enable correlation thereof.

In accordance with a further aspect of the invention, the apparatus comprises means for selective activation of a sensing device by shining an emitted light beam at the sensing device.

In accordance with a further aspect of the invention, the apparatus comprises wireless means for directing a sensing device to use a selected radio channel to transmit the environmental data in a multi-sensing device use scenario.

In accordance with a further aspect of the invention, the apparatus comprises wireless means for directing a sensing device to emit sound, either audible or inaudible, to help an operator in differentiating the sensing device from real produce/article goods being processed or handled.

In accordance with yet another aspect of the invention, the apparatus comprises a receiver device for interfacing a sensing device and a display device for instant real-time display of environmental measurements.

The advantages are derived from a configurable and customizable apparatus for monitoring, detection, measurement, and transmission of environmental conditions experienced by produce and article goods in processing and handling thereof. The monitoring, detection, measurement and gathering of the environmental conditions data is found to be important in preventative maintenance, handling efficiency and performance monitoring in food safety and quality programs. The apparatus provides handlers with directed information on what processing and handling issues to address to recover revenues previously accepted as lost product and packaging.

### BRIEF DESCRIPTION OF THE DRAWINGS

The features and advantages of the invention will become more apparent from the following detailed description of the preferred embodiment(s) with reference to the attached diagrams wherein:

FIG. 1 is a schematic diagram showing elements implementing the exemplary real-time wireless detection, measurement and transmission apparatus in accordance with an exemplary embodiment of the invention;

FIG. 2 is a schematic diagram showing the real-time wireless detection, measurement, and transmission apparatus exemplary used to inspect a goods processing line, in accordance with the exemplary embodiment of the invention; and

FIG. 3 is a schematic diagram showing the real-time wireless detection, measurement, and transmission apparatus exemplary used to inspect a goods in transport, in accordance with the exemplary embodiment of the invention.

It will be noted that in the attached diagrams like features bear similar labels.

### DETAILED DESCRIPTION OF THE EMBODIMENTS

There is a strong growing need for remote diagnostic tools for instant real-time detection of various environmental factors affecting produce and/or goods in handling and/or processing environments. In particular, there is a need for continuous real-time detection, profiling, analysis, and reporting of multiple environmental conditions experienced simultaneously.

In accordance with an exemplary embodiment of the invention, manufacturers, producers, handlers, etc. are provided with means for dynamic monitoring produce/goods, simultaneously, in respect of a multitude of significant environmental parameters, during the handling, processing, and storage of produce/goods.

Some of the measured environmental parameters, that may be deemed important to monitor in respect of particular applications, include: conductivity, humidity, impact, pH, pressure, strain, temperature, position, orientation, roll, angular momentum, incident light intensity, etc. Measurements can be performed under ideal conditions, as well in hostile and potentially damaging processing/handling environments, where wireless transmission of the multiple environmental measurement and analysis data enables: preventative maintenance of processing/handling equipment, improvement in the handling efficiency of produce/goods, performance monitoring in effecting food safety and quality assurance programs, etc.

In accordance with the exemplary embodiment of the invention, the apparatus presented herein below provides real-time measurement, analysis, and wireless transmission, of environmental parameters and analysis data while the apparatus is positioned in a similar manner to that of an actual monitored article (produce/goods): pallet, container, vessel, or even a produce replica; during processing, storage, handling, transport, etc.

Dynamic temperature measurement in the context of product monitoring has a large number of applications. Temperature variations can have a negative effect on product quality, food safety, consumer safety, etc. Also correct exposure to temperature cycles ensures destruction of micro-organisms in retaining produce freshness, as well provides vial sterilization.

In accordance with the exemplary embodiment of the invention, derived monitored environmental parameters are determined from a multitude of measured environmental parameters. Determining derived monitored environmental parameters may be profiled, analyzed, and reported; and include for example: determining angular moments imparted from experienced multi-directional impact measurements, determining dew point determination from experienced ambient temperature, humidity, and pressure measurements, etc. An exemplary application where continuous dew point determination is useful, is the storage, handling, and transport of potatoes, which when wet, undesirably start to sprout.

In accordance with the exemplary embodiment of the invention, a standalone environmental parameter measurement and reporting device, referred to as a sensing device for short, includes a custom molded enclosing housing into which measurement, analysis, and reporting means are housed and mechanically fastened thereto. Each sensing device is a customized package including: group of measurement sensors, customized measurement analysis electronics, and a transceiver enclosed in an exact facsimile of a target produce/good article, such as, but not limited to: an egg, kiwi, vial, can, bottle, etc.

In accordance with the exemplary embodiment of the invention, steps are taken to ensure that each one of the multiple sensors employed, in respect of a particular application, is positioned within the sensing device to measure the full effect of the corresponding monitored environmental parameter. The sensing device is co-located with actual produce/goods to experience measured environmental conditions through all stages of processing, packaging, storage, shipping, etc. as the produce/goods.

To provide external ambient temperature monitoring, the enclosure, in the form of one of the monitored articles, has a removable temperature probe that can be changed dependent on application (processing, transport, storage, etc.) to ensure temperature measurement accuracy as different temperature ranges are typically encountered depending on the application. The enclosing housing has a hole and a grommet placed about the hole. A temperature probe is inserted through the grommet forming a water tight seal therewith. The temperature sensor is located within the probe located just below the exterior surface of the temperature probe to avoid damage thereof and to measure temperature as experienced by the actual product/good in situ.

Similar mounting provisions are made for other sensors i.e. humidity, pH, conductivity, etc. so these may be installed and positioned as to experience the monitored environmental condition directly.

It was mentioned that imparted angular moment measurements are derived from impact measurements. Therefore, the location where impact is measured is very important. The solution described in the above referenced U.S. Pat. No. 6,125,686, is a single-sensor solution employing a single tri-axial impact sensor package. The single tri-axial impact sensor package, although providing a reduction in the complexity of the electronics package by requiring a single sensor port interface, was found to be impractical as only unidirectional impact measurements were provided and the tri-axial sensor package itself was too bulky, limiting its use to monitoring large produce and goods. The large size of the sensor package did not allow correct positioning thereof for all applications and therefore did not allow correct impact measurement particularly in respect of small produce/goods.

In order to achieve correct impact measurements and to derive correct imparted angular moments, multiple small bidirectional single-axe impact sensors are employed.

In accordance with the exemplary embodiment of the invention, in order to measure impact, the housing is designed, and the position of the electronics within is selected, such that the sensor device has a mass distribution which mimics the mass distribution of the monitored produce/good. Three small bidirectional single-axe impact sensors are positioned at/about the center of gravity of the sensing device. Additional impact sensors are placed at positions away from the center of gravity of the device, measurements from the multiple impact sensors are combined to determine imparted angular moments. The impact sensors may include accelerometers.

In accordance with an exemplary implementation of an impact sensor, an accelerometer measuring acceleration caused by force pushing or pulling on the surface of a piezo-electric crystal is used. The piezo-electric crystal produces an electric charge/potential across thereof depending on the amount, and direction, of the force exerted on the surface thereof. The electric charge/potential is amplified and post-processed by the electronics package to produce a voltage or a current output. The use of piezo-electric crystal devices provides enhanced accuracy and reliability at a reduced foot print and cost when compared to prior art beam-bending implementations.

The sensing device is serviceable, which is made possible by the design of the enclosure. For example, the enclosure may include multiple parts that engage together to provide a watertight enclosure. A first main part of the enclosure is typically hollow and houses the electronics package, and a threaded battery lid (second part) allows the battery (the power source) to be changed easily, and without disturbing the electronics package (as typically the sensors are calibrated and should not be disturbed). A threaded retaining ring may be employed to secure the electronics package. A variety of retaining means may be employed without limiting the invention thereto.

In the case of monitoring produce, such as pineapples and/or kiwi, the enclosure may comprise of acrylic parts, possibly with a urethane coating simulating the surface texture and density thereof.

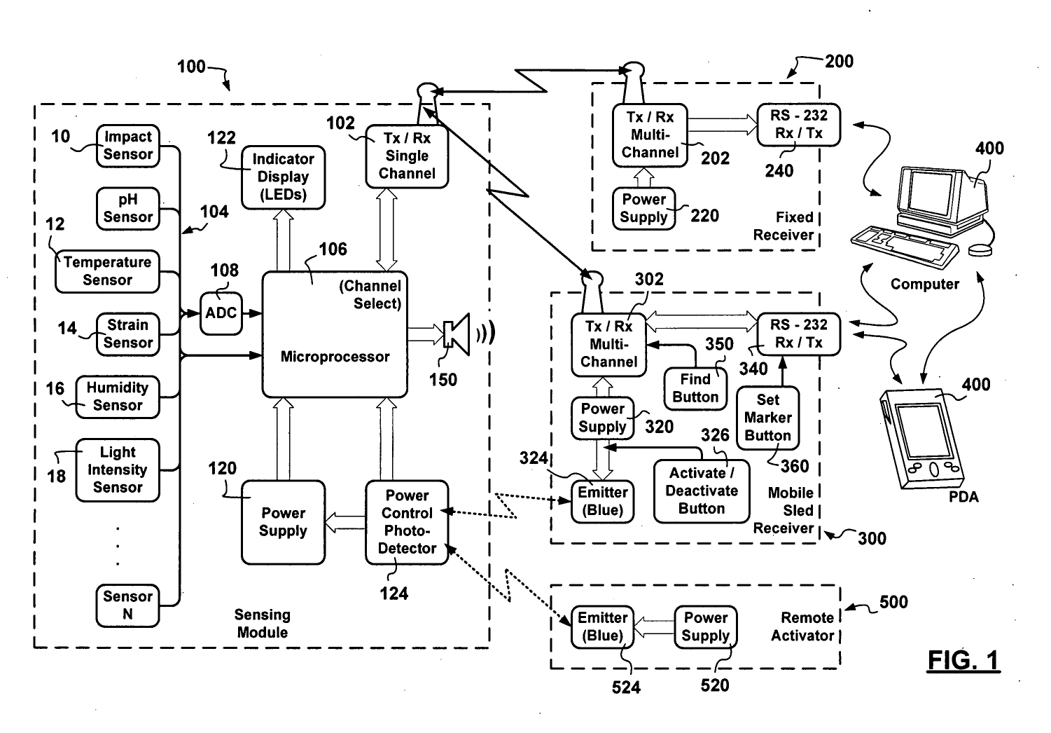

Making reference to FIG. 1, the electronic components of the sensing device 100 may be correspondingly divided into two groups. Each group of electronic components may be connected on a printed circuit board.

The first printed circuit board, referred to herein as the main board, includes the following:

transceiver 102 to transmit the data to a remote transceiver 202/302. The transceiver 102, typically, but not limited to, a single channel transceiver, can be set to transmit on various different frequency bands so that several sensing devices 100 can be operated concurrently in a monitoring area without interfering with each other. Multiple sensing devices 100 can transmit to measurement data to several receiving modules 200/300 simultaneously or to a single receiver 200/300 equipped with multi-channel receiving capabilities 202/302. Status and performance information may also be collected and transmitted periodically;

a plurality of sensor interfaces 104. Individual accelerometers/impact sensors 10 may be employed to accurately and independently measure negative and positive impacts on each orthogonal axis at various positions with respect to the sensing device 100. Temperature 12, strain 14, humidity 16, incident light intensity 18, etc. sensors connect to corresponding sensor interfaces 104; and

a microprocessor 106 is programmed (with firmware/software) to: read all sensors 104, perform software processing on the measured data, and issue information for transmission through the on-board radio transceiver 102. The firmware/software executed by the microprocessor 106 can be upgraded “in circuit”, typically via the on-board transceiver 102 without limiting the invention thereto. Wireless means for upgrading the firmware/software reduce tampering with the sensing device 100 and the calibrated sensors.

The microprocessor 106 collects the measurement data from each of the individual sensors at a rate dependent on the number of sensors. Reading sensor output at high rates ensures the detection of short-duration changes. Every sensor has a settling rate which determines the upper limit at which meaningful sensor output can be obtained. In practice reading the sensors at 5 to 10 KHz is adequate for most applications.

A multitude of microprocessors 106 may be used, and the selection is left to design choice: some microprocessors 106 include additional on-chip functions while others have faster processing capabilities, also cost plays an important role. Sensor output being analog, as mentioned above, has to be digitized for processing and/or transmission by the sensing device 100. As such, analog-to-digital conversion may be provided by a separate analog-to-digital converter 108 or the microprocessor 106 may include analog-to-digital conversion functionality.

Measurements are collected continuously at the collection rate. The measurement data for each sensor may be conveyed as a corresponding continuous stream of measurement values for profiling. Also the measurement data may be subjected to at least threshold to derive alarm information therefrom. Subjecting the measurement data to the at least one threshold may implemented in a variety of ways in hardware or in software. Again design choice is employed in implementing thereof. Software methods are typically chosen as stringent requirements are imposed on the foot print of the electronics package inside the housing of the sensing device 100.

A related measurement data processing function is know as peak detection. Peak detection may be used both, in raising alarms when a particular sensor output is above/below a sensor output level, in auto-calibration, and in auto-ranging.

The microprocessor 106 may perform a software peak-detection operation on each channel individually, then transmits the peak data through the radio transceiver 102. In practice, performing peak detection at a rate of 32 to 40 Hz is found to be adequate in most applications. The transmitted data includes an actual peak value for each individual sensor since the last transmission, as well timestamp information associated with the transmission. Peak detection data, which can be used to raise alarms, may be sent separate from measurement data streams used in profiling.

When the peak detection processing is used auto-ranging, peak detection information is provided to a gain control circuit that allows measurements to enable a more precise digital expression when measuring low-amplitude sensor output as well high-amplitude sensor output which can change very rapidly. Auto-calibration is similar to auto-ranging functionality in that measurement data processing ensuring reduced sensor drift. Particularly, a high-resolution calibration-free temperature sensing device 100 can be implemented.

The sensing device 100 may also include a software/hardware-based mechanism for turning off the sensing device 100 if no meaningful measurements have been collected for a predetermined length of time at the highest gain factor, conserving battery life.

The bidirectional radio communications function 102/202/302 enables remotely changing operating modes of the sensing device 100 including changing the transmission radio frequency mentioned above and for remotely changing auto-power down delay. This facility can also be used to query the sensing device for status and diagnostic information also mentioned above.

The second printed circuit board, referred to herein as the battery board, contains the remaining electronics. The battery board is typically housed in the battery lid apart from the sensing electronics to ensure that the calibrated sensors are not disturbed in replacing the battery. The battery board has battery clips to connect the battery (power supply) 120 having power conversion circuitry if necessary. The power conversion circuitry converts battery voltage to different voltage outputs used to power the sensors, microprocessor 106, transceiver 102, etc. It is envisioned that some applications may only require only a small amount of power and therefore solar cells could provide the necessary power. At least one light emitting diode 122 represents an indicator display indicating power and sensing module status.

A photo sensing device 124 may be used in remotely activating the sensing device 100 when the radio transceiver 102 is turned off to conserve power. Therefore the photo sensing device 124 detects a specific wavelength of light and turns the power to the unit on and off remotely, without the need to physically handle the sensing device 100, and without affecting the monitoring environment. A clear window may be provided in the sensing device housing for the photo sensing device 124 allow non-intrusive operation of the sensing device 100. Provisioning a completely enclosed sensing device 100 improves validation and life time of the solution.

Without departing from the spirit of the invention, the printed circuit boards themselves may be used for providing reinforcing strength when the enclosure itself, due to small size requirements cannot be reinforced, the printed circuit boards may be soldered together at angles forming a rigid structure to which the sensors are attached to ensure correct positioning. In such implementations, due to space restrictions, care is to exercised not to disturb the calibrated sensors in replacing the battery.

A mechanism may be provided on the circuit boards enabling automated testing both during manufacturing of the sensing device 100 as well during field servicing of the sensing device 100. The automatic testing mechanism may include test pins and/or wireless test functionality.

Further, the electronics package may include a sound-based, either human-audible or ultrasonic, means 150 of locating the sensing device 100.

The sensing device 100 does not typically store measurement data collected in the sensing unit. All data is transmitted via a wireless link for instant real-time reporting. However, applications in which processing steps such as sterilization are performed, may require temporary storage of sensor output measurements in local storage (not shown). For example, autoclaves have metallic chambers which may not permit radio transmission from within. Some microprocessors 106 have ample on-chip storage, but come at relatively high costs.

The measurement data transmitted by the sensing device 100 is received by a remote receiver. Two types of receivers are shown in FIG. 1. The first is a fixed receiver 200 typically connected to a computer. The fixed receiver 200 simply relays all transmissions from the sensing device 100 to the computer 400, perhaps providing adaptation functionality exemplary conveying received information over a serial link. The use of fixed receivers 200 enables the monitoring of a long processing line from multiple locations along the processing line using a single computer. The second receiver type is known as a mobile “sled” 300 adapted to be connected to a handheld computing/display device 400. The mobile sled 300 itself may include:

a power supply 320 typically running off of battery power, or a wall adapter;

transceiver 302 receiving the information from the sensing device 100; and

an communications port 340 (RS232, Universal Serial Bus, Fire Wire, etc.) converting received information for transmission to a display or recording device.

The mobile sled 300 may also contain a circuit means enabling detection and output sending device status, and to control the sensing device 100. The circuit means would include a combination of:

battery or power supply status indicators (not shown), sound based alerting means (not shown) may be also be employed to alert the user to operating issues;

a sensing device find button 350 which operable to activate the sound emitter 150 on the sensing device 100.

a sound-based detection circuit (not shown) enabling locating the sensing device 100 when the sound emitter 150 on the sensing device 100 emits ultrasonic waves so as not to disturb the environment; and

light-based means 324 for turning the sensing device 100 on or off. A button 326 or a software-controlled switch activates a light source 324 emitting light at a specific wavelength detected by the photo sensing device 124 of the sensing device 100.

The mobile sled 300 typically does not store reported information, acting as a gateway to the display device 400. Applications such as long distance transport monitoring may require large amount of data storage in which case the mobile sled 300 may include a data store (not shown).

The display device 400 may include any off the shelf such as a laptop (400), a Personal Digital Assistant (PDA), laptop, or desktop computer. The display device 400 would execute software enabling the display of sensor output measurement data, reported derived information, and locally derived information, in real-time as events happen, in a useful and meaningful way to the operator of the unit. The combined information displayed would be particular to customer requirements and the particular application including any combination of the sensor output.

Other software functions may include playback and profile comparison, data analysis and statistics measurements on user-selectable portions of the reported data.

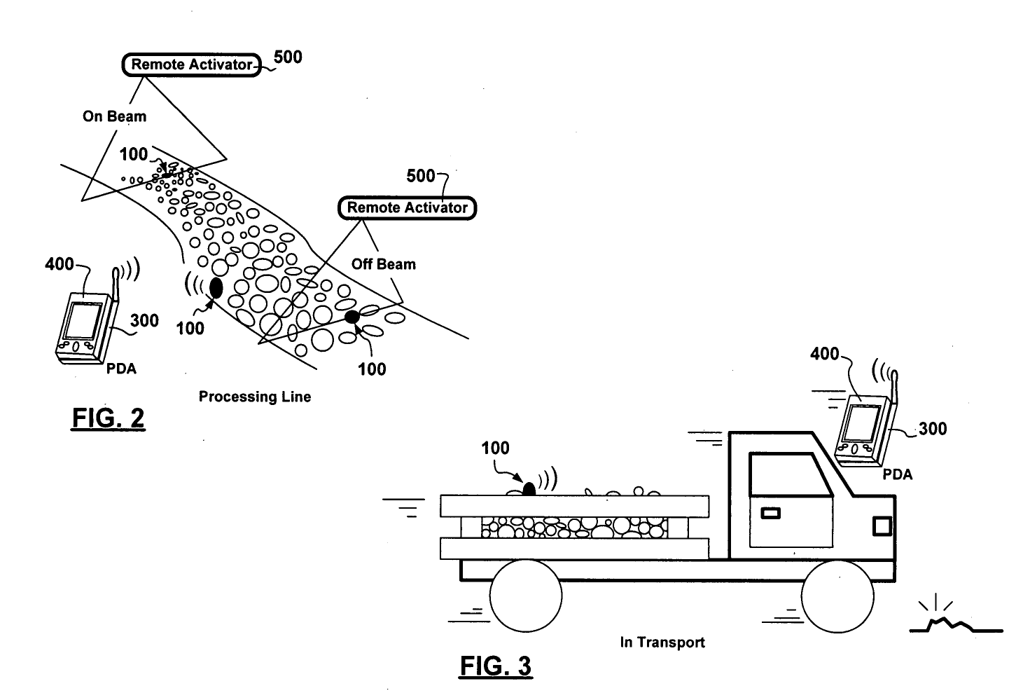

Details of a remote activator module 500 are provided in FIG. 1. The remote activator module 500 includes a power supply 520 and a light source 524 emitting light at a specific wavelength detected by the photo sensing device 124 of the sensing device 100. In accordance with an exemplary implementation an on remote activation module 500 is used to specify the beginning of a monitoring portion of a processing line and another off remote activation module 500 is used to specify the end of the monitoring portion of the processing line, as shown in FIG. 2.

In accordance with an exemplary implementation, a handheld PDA device 400 with a mobile sled 300 is shown in FIG. 3, is used for instant real-time display and monitoring of environmental parameter measured data while in transport. A driver is enabled to enter markers into the data stream, for example specifying “pavement bump near mile stone number.”. Location information may also be provided by employing a Global Positioning System (GPS) sensor. Following produce/goods transport, the recorded measurement data may be read out from the PDA memory over a data port associated therewith, to an external device, such as the computer 400 shown in FIG. 1, for further processing.

Real time interaction between an operator and the display device 400 in response to real-time received data allows comparisons and event tracking for easy determination of areas of concern. These functions are also useful in quality control applications.

In accordance with the exemplary embodiment of the invention, all measurements are stored. In particular capturing and storing low-level measurement data helps detect problems with processing and handling equipment before these become serious enough to cause product damage. For example, at a manufacturing plant using beverage can sterilization equipment, a sensing device 100 shaped in the shape of a can, is used repeatedly in the sterilization processing lines. Each sterilizer has an operational “signature” which depends on the mechanical design of the unit. Periodic recording of sterilizer signatures may be used to determine if continued use of a sterilizer is causing undue wear on cans or other problems, long before can damage is experienced.

In accordance with the exemplary embodiment of the invention, a real-time monitoring and display of reported environmental conditions is provided. An operator inspecting a processing line, is enabled by actuating buttons associated with the mobile sled receiver module 300 or the PDA 400 to insert markers in the data stream in real time as the received data is displayed to the operator in real time as shown in FIG. 2 and FIG. 3. This greatly improves the ability of the operator to correlate the data with the location where the data was reported from. The sled module 300 may also include a marker set button 360 for this purpose.

The following are exemplary implementations:

Each year the international bottling industry loses millions of dollars on handling abnormalities, line changeovers and production line shutdowns. An exemplary sensing device 100 shaped as a bottle is instrumented with impact sensors to measure vertical and horizontal impact imparted to a glass container at impact points located at the shoulder and the base thereof as impact profiles will differ at different locations. Distinguishing the different impact profiles at the shoulder and heel of a 4-10 inch tall by 1-3 inch diameter bottle will determine in more detail which processing machinery imparts excessive impacts.

The exemplary impact measuring sensing device 100 in the shape of a bottle may make use of 5 piezo-electric accelerometers to measure impact at the shoulder and heel of the bottle separately from, and in addition to, the overall impact measured at described above. An exemplary arrangement of individual impact sensors 10 includes: three orthogonal impact sensors 10 at base or heel of the bottle shaped sensing device 100 measuring impact in two horizontal directions (X & Y) as well in the longitudinal or vertical direction (Z); two impact sensors 10 at the shoulder of the bottle shaped sensing device 100 measuring impact in the horizontal directions (X & Y). Additional impact sensors 10 can be used depending on the needs of the customer and the application.

The bottle shaped sensing device 100 can provide bottling plant managers with the ability to instantly view the handling and performance characteristics of individual packaging lines from the perspective of a bottle itself. The bottle shaped sensing device 100 runs directly through the bottling processing line alongside real bottles, identifying excessive impact points reporting impact location and magnitude instantly in real-time. The gathered information enables bottling plant operators to improve efficiency in bottle packaging, reduces incidents of bottle scuffing and fracture, improves line changeover efficiencies, and aids in daily preventative maintenance.

Different impact profiles at different locations are experienced not only by bottles, but also by large articles such as large produce (pineapples, melons, etc.)

In the food processing industry, ensuring limited exposure to pressure helps prevent a variety of aspects of processing and handling including: label scuffing, container failure, can popping, etc.

An exemplary implementation of a sensing device 100 in the shape of a can, is employed to detect in-line can abuse linked to unnecessary spoilage, shrinkage, and food safety risks. Can abuse causes rim and wall denting ultimately transferring moisture and bacteria from machinery to food or beverage sealed therein. Prior art methods of measuring temperature require lengthy test times, multiple systems, and do not provide instant feedback during the pasteurization process.

A can shaped sensing device 100 is inserted directly into handling machinery to experience the hostile environments a typical can is subjected to for preventative maintenance and daily checks. The can shaped sensing device 100 monitors food can handling in packaging lines and inside retort pasteurizers to identify can denting, ambient temperature and can rotation, by running directly through the processing systems alongside real cans.

In the agricultural industry, at worst, a broken egg is worthless; and a cracked egg, if it can be sold at all, is worth only a fraction of its unblemished value. It is therefore critical to keep all losses of shell integrity to an absolute minimum from the moment the egg is laid. Losses are categorized in two ways, mechanical cracks or breaks and internal defects such as bloodspot, over which the producer or packer has no control. Damage levels of 7-10% are reported to occur.

The exemplary an egg shaped sensing device 100 designed to mimic real egg dimensions, is used to instantaneously detect harmful aspects of egg handling operations. The egg shaped sensing device 100 runs directly through the processing system alongside real eggs, identifying abuse points, reporting location and magnitude of abuse instantly in real-time. The user is enabled to determine immediately the effect mal-adjusted equipment has on the eggs: where the abuse occurs, and if in fact the abuse is problematic and requires attention. The egg shaped sensing device also reports temperature extremes which could affect the freshness and eventually spoil eggs.

The embodiments and implementations presented are exemplary only and persons skilled in the art would appreciate that variations to the above described embodiments and implementations may be made without departing from the spirit of the invention. The scope of the invention is solely defined by the appended claims.

I claim:

1. A standalone environmental parameter measurement and reporting device for use in monitoring the environmental conditions to which a plurality of articles are subjected, said device being subjected to the same environmental conditions as said articles, the device comprising a housing designed to be embedded with said articles, the device further comprising:
a. a plurality of sensors for monitoring different environmental conditions, each sensor providing a sensor output based on a monitored environmental condition experienced;
b. a processor deriving at least one environmental parameter value from a plurality of sensor output measurements obtained from the plurality of sensors; and
c. a transceiver for reporting said derived environmental parameter value to a remote receiver.

2. The device claimed in claim 1, wherein the housing further comprises at least one grommetted hole therein enabling sensor exposure to the monitored environment surrounding the device while providing sealing between the sensor and the housing.

3. The device claimed in claim 1, wherein the housing further mimics monitored articles including one of: a product and a durable good, in respect of one of: shape, surface texture, surface physical properties, and mass distribution.

4. The device claimed in claim 3, wherein the housing further comprises:
a. a first housing portion the plurality of sensors, the processor, and the transceiver;
b. a second housing portion housing a power source; and
c. retaining means providing sealed engagement between the first and second housing portions.

5. The device claimed in claim 3, wherein one of strength and rigidity is provided by a plurality of printed circuit boards soldered together at angles to each other.

6. The device claimed in claim 3, wherein each sensor comprises one of: an impact sensor, a pH sensor, a temperature sensor, a strain sensor, a humidity sensor, a light intensity sensor, a position sensor, an orientation sensor, a roll sensor, an acceleration sensor, and a conductivity sensor.

7. The device claimed in claim 6, wherein the impact sensor includes a piezo-electric sensor.

8. The device claimed in claim 6, wherein monitoring impact experienced by the device, the device further comprises: three single-axe bidirectional impact sensors orthogonally oriented with respect to each other and situated about the center of mass of the device.

9. The device claimed in claim 1, wherein at least one sensor comprises a removable sensor connected to a corresponding sensor port interface associated the processor.

10. The device claimed in claim 1, wherein derived parameter values comprise one of: an angular moment imparted to the device, and a dew point.

11. The device claimed in claim 10, wherein monitoring angular moment imparted to the device, the device further comprises: a plurality of impact sensors situated away from the center of mass of the device.

12. The device claimed in claim 1, wherein the transceiver further comprises: a single channel transceiver, transmitting on a selectable frequency band, enabling concurrent use of the device with other devices in a single monitoring area.

13. The device claimed in claim 1, further comprising one of: hardware analog-to-digital sensor output converter, and a software analog-to-digital sensor output converter.

14. The device claimed in claim 1, further comprising an auto-gain control circuit providing one of: auto-calibration and auto-ranging.

15. The device claimed in claim 1, further comprising a power conversion circuit converting power source voltage to a plurality of voltage outputs, each voltage output being used to provide power to one of: a sensor, the processor, and the transceiver.

16. The device claimed in claim 1, further comprising at least one light emitting diode indicating device status without affecting the monitored environment.

17. The device claimed in claim 1, further comprising a photo device enabling remote activation of the device without affecting the monitored environment.

18. The device claimed in claim 1, further comprising a sound emitter, emitting one of: human-audible sound and ultrasound, to aid in locating the device.

19. The device claimed in claim 1, optionally comprising a temporary data storage enabling monitoring environmental conditions in applications in which the device is temporarily shielded preventing radio transmission.

20. The device claimed in claim 1, further comprising one of wired and wireless self-testing mechanism.

21. A receiving module, the receiving module comprising:
a. a multi-channel transceiver for receiving at least one environmental parameter value and transmitting at least one control command; and
b. communication port for relaying the at least one environmental parameter value.

22. The receiving module claimed in claim 21, comprising one of: a fixed receiver and a mobile receiver.

23. The receiving module claimed in claim 21, further comprising one of a: sensing device activate button, sensing device find button, and a marker set button.

24. The receiving module claimed in claim 21, further comprising a light emitter for actuating a sensing device.

25. The receiving module claimed in claim 21, further comprising a sound detection circuit used in locating an ultrasound emitting sensing device.

26. A method of monitoring environmental conditions experienced by a plurality of articles during one of handing, processing, storage, and transport, a monitoring device being subjected to the same environmental conditions as said articles, the device comprising a housing designed to be embedded with said articles, the method comprising steps of:
a. obtaining a plurality of measurement values from a corresponding plurality of sensors; and
b. deriving at least one environmental parameter value from the plurality of measurement values obtained.

27. The method claimed in claim 26, wherein obtaining the plurality of measurement values, the method further comprises a step of collecting sensor measurements continuously at a collection rate.

28. The method claimed in claim 27, wherein the collection rate is between 5 and 10 KHz.

29. The method claimed in claim 26, wherein deriving the at least one environmental parameter value, the method comprises performing calculations in one of: hardware and software.

30. The method claimed in claim 26, wherein deriving the at least one environmental parameter value, the method comprises a step of performing peak detection on one of: successive measurement values and successive derived environmental parameter values.

31. The method claimed in claim 30, wherein peak detection is performed at a rate between 32 and 40 Hz.

32. The method claimed in claim 26, further comprising: subjecting stream of derived environmental parameter values to a peak detection step determining whether the derived environmental parameter value is above a threshold level.

33. The method claimed in claim 32, further comprising a step of: performing one of auto-calibration and auto-ranging based on the result of the peak detection step.

34. The method claimed in claim 26, further comprising a step of transmitting the at least one derived environmental parameter value.

35. The method claimed in claim 34, wherein prior to transmitting the at least one derived environmental parameter value, the method further comprises a step of selecting a transmission frequency band.

36. The method claimed in claim 35, wherein selecting the transmission frequency band, the method further comprises a step of receiving a command to select a transmission frequency band.

37. The method claimed in claim 26, further comprising a subsequent step of: inserting a marker between one of: successive measurement values and successive derived environmental parameter values.

38. The method claimed in claim 26, further comprising optional subsequent steps of: performing a measurement value profile comparison, and extracting a statistic value in respect of a selected group of derived environmental parameter values.

39. The method claimed in claim 26, further comprising steps of:
a. receiving a request for status reporting;
b. providing a status report; and
c. transmitting the status report.
